(19) United States
(12) Patent Application Publication
(10) Pub. No.: US 2023/0399697 A1
(43) Pub. Date: Dec. 14, 2023

(54) ASSESSING RISK WITH PRE-OPERATIVE TOTAL CELL-FREE DNA

(71) Applicant: The Medical College of Wisconsin, Inc., Milwaukee, WI (US)

(72) Inventors: Aoy Tomita Mitchell, Elm Grove, WI (US); Michael Mitchell, Elm Grove, WI (US)

(21) Appl. No.: 18/035,921

(22) PCT Filed: Nov. 9, 2021
(86) PCT No.: PCT/US2021/058605

§ 371 Date: May 8, 2023

Related U.S. Application Data
(60) Provisional application No. 63/111,593, filed on Nov. 9, 2020.

(51) Int. Cl. C12Q 1/6883

(52) U.S. Cl. CPC C12Q 1/6883; C12Q 2600/118; C12Q 2600/106

(57) ABSTRACT

This invention relates to methods and compositions for assessing risk based on amount(s) of total cell-free DNA in a subject, such as including at least one pre-operative sample from the subject.

### RELATED APPLICATION

This application claims the benefit of priority under 35 U.S.C. § 119(e) to U.S. Provisional Application, U.S. Ser. No. 63/111,593, filed Nov. 9, 2020, the contents of which are incorporated by reference herein in their entirety.

### FIELD OF THE INVENTION

This invention relates to methods and related compositions for assessing a total amount of cell-free nucleic acids, such as cell-free DNA (cf-DNA), in a sample from a subject prior to surgery. The methods and compositions provided herein can be used to risk post-operative in a subject, in some embodiments.

### SUMMARY OF INVENTION

The present disclosure is based, at least in part on the surprising discovery that pre-operative total cf-DNA can be a marker of risk, such as of death or adverse events, need for mechanical support, etc., post-operative in a subject. In any one of the methods provided herein, the subject is one who will undergo cardiac surgery, cardiothoracic surgery, congenital heart surgery, such as that associated with cardiopulmonary bypass (CPB), etc. In any one of the methods provided herein, the subject is a pediatric subject, such as one undergoing cardiac surgery, cardiothoracic surgery, congenital heart surgery (in some embodiments, associated with CPB). In any one of the methods provided herein, the subject is any one of the subjects described herein.

Therefore, methods of obtaining amount(s) of pre-operative total cf-DNA, which can be obtained as ng/mL in any one of the methods provided herein, are provided herein whereby the amount(s) can be used to assess the risk to a subject and/or assess any one of the foregoing. Any one of the methods provided herein can be used for such purposes.

Provided herein are methods related to obtaining amount(s) of pre-operative total cf-DNA at one or more points in time. Also provided are related reports, kits, databases, compositions, etc. related to such determinations and/or including such amount(s) alone in combination with threshold value(s) or other amount(s), such as other amount(s) obtained from other points in time. Provided herein are ways and related aspects of monitoring the health of a subject over time with total cf-DNA as a biomarker.

In any one of the methods provided herein, the threshold is any one of the thresholds provided herein. In one embodiment of any one of the methods provided herein, the threshold is 17 (e.g., 17.1), 18, 19 or 20 ng/mL.

In any one of the methods provided herein, the method comprises treating the subject or suggesting a treatment to a subject based on the assessed risk. In one embodiment of any one of the methods provided herein, the treating can comprise an alternative treatment, an intensified treatment and/or earlier treatment of the subject. In one embodiment of any one of the methods provided herein, the treating can comprise delaying or canceling the surgery (or alternatively, performing surgery earlier than scheduled).

In any one of the methods provided herein, the method comprises monitoring the total cf-DNA amount in the subject over time and can comprise earlier monitoring and/or increased frequency of monitoring the subject based on the assessed risk.

In some aspects, the disclosure provides method of assessing a risk in a subject, the method comprising: obtaining an amount of pre-operative total cell-free DNA (cf-DNA) in a sample from the subject; and optionally, reporting and/or recording the amount of pre-operative total cf-DNA.

In any one of the embodiments provided herein, the method further comprises comparing the amount of pre-operative total cf-DNA to a threshold total cf-DNA value or other amount from a different point in time.

In any one of the embodiments provided herein, the method further comprises determining a risk in the subject based on the obtained amount of pre-operative total cf-DNA or the comparison to the threshold or other total cf-DNA value.

In any one of the embodiments provided herein, the method further comprises obtaining an amount of pre-operative total cf-DNA in one or more additional samples from the subject, each taken at different point in time.

In some embodiments, the different point(s) in time are one or more of any of the different points in time provided herein. In some embodiments, the method further comprises comparing the amount(s) of pre-operative total cf-DNA to threshold values or amounts from one or more different points as described above. In some embodiments, the method further comprises determining a risk in the subject by making the comparison.

In any one of the embodiments provided herein, the method further comprises obtaining an amount of post-operative total cf-DNA in one or more additional samples from the subject, each taken at different point in time.

In some embodiments, the post-operative different point(s) in time are one or more of any of the different points in time provided herein, such as one or more or all of the following post-operative times: immediately following surgery, 12 hours, 24 hours, 48 hours, 72 hours, 120 hours, 168 hours, 10 days, 14 days, 21 days, or 28 days. In some embodiments, the method further comprises comparing the amount(s) of post-operative total cf-DNA to threshold values or amounts from one or more prior points in time. In some embodiments, the method further comprises determining a risk in the subject based on a comparison(s) described above.

In some embodiments, the method further comprises determining a treatment or monitoring regimen for the subject based on the amount(s) of pre-operative total cf-DNA and/or post-operative total cf-DNA and/or the comparison(s).

In some embodiments, the pre-operative total cf-DNA and/or post-operative total cf-DNA is obtained from samples taken from the subject once, twice, or thrice daily.

In some embodiments, the subject is a pediatric subject. In some embodiments, the subject is assessed for at least 1, 2, 3, 4, 5, 6, or 7 days prior to surgery. In some embodiments, the method further comprises providing or obtaining one or more threshold values or amount(s) from one or more different points in time.

In some embodiments, a threshold value is provided for each time point an amount of pre-operative total cf-DNA and/or post-operative total cf-DNA is obtained. In some embodiments, the method further comprises obtaining the sample(s) from the subject.

In some embodiments, the amount(s) and/or threshold value(s) are provided in a report. The disclosure provides, in some embodiments, a report that comprises the amount(s) and/or threshold value(s) of any one of the methods described herein. In some embodiments, the amount(s) and/or threshold value(s) are recorded in a database. The disclosure provides, in some embodiments, a database that comprises the amount(s) and/or threshold value(s) of any one of the methods described herein.

In some embodiments, the determining a monitoring regimen comprises obtaining amount(s) of total cf-DNA in the subject over time or at a subsequent point in time, or suggesting such monitoring to the subject. In some embodiments, the time between samples is decreased if the amount(s) of pre-operative total cf-DNA and/or post-operative total cf-DNA is increased relative to threshold(s) or amount(s) from earlier time point(s). In some embodiments, the time between samples is increased if the amount of pre-operative total cf-DNA and/or post-operative total cf-DNA is decreased relative to threshold(s) or amount(s) from earlier time point(s). In some embodiments, the determining a monitoring regimen comprises using or suggesting the use of one or more additional test(s) to assess the subject. In some embodiments, the determining a monitoring regimen comprises assessing or suggesting the assessment of the subject at one or more points in time, determining or suggesting the frequency or timing of the assessment, changing the frequency or timing of the assessment or suggesting such change.

In some embodiments, the determining a treatment regimen comprises selecting or suggesting a treatment for the subject or changing the treatment of the subject or suggesting such change or changing the timing or frequency of the treatment of the subject or suggesting such change. In some embodiments, the determining a treatment regimen comprises treating the subject. In some embodiments, the determining a treatment regimen comprises providing information about a treatment to the subject.

In some embodiments, the sample is a blood, plasma or serum sample. In some embodiments, the subject is a non-transplant subject. In some embodiments, the subject is as cardiac surgery subject. In some embodiments, the subject is any one of the subjects provided herein.

In some embodiments, the risk is of any one of the states or conditions or outcomes as provided herein. In some embodiments, the risk is a risk of cardiac arrest, need for mechanical ventilation, or death following surgery.

In some embodiments, the threshold(s) is/are any one of the threshold value(s) as provided herein, such as 15, 16, 17 (e.g., 17.1), 18, 19, or 20 ng/mL.

In some embodiments, the subject is monitored over time, such as with any one of the monitoring regimens as provided herein. In some embodiments, the subject is treated or treated over time, such as with any one of the treatments provided herein. In some embodiments, the treatment or monitoring regimen is suggested to, prescribed for, given to or performed on the subject.

Any one of the methods provided herein can include any one or more (or combination thereof) of the steps of embodiments provided herein.

### BRIEF DESCRIPTION OF FIGURES

The accompanying figures are not intended to be drawn to scale. The figures are illustrative only and are not required for enablement of the disclosure.

FIG. 1 illustrates an example of a computer system with which some embodiments may operate.

FIG. 2 shows total cell-free DNA (tcf) in samples taken at the timepoints indicated on the X-axis in pediatric subjects following congenital heart surgery with cardiopulmonary bypass (CPB).

FIG. 3 shows total cf-DNA and outcome in pediatric subjects following congenital heart surgery with CPB. CAED represents subjects experiencing post-operative cardiac arrest, ECMO, and/or death (CAED).

FIG. 4 shows an experimental of the correlation between pre-surgery total cf-DNA and CAED in pediatric subjects following congenital heart surgery with CPB.

### DETAILED DESCRIPTION OF THE INVENTION

Pre-operative risk stratification in congenital cardiac surgery includes patient- and procedure-related factors, which may be used in clinical decision making as well as program performance evaluation. Despite these tools, unidentified factors contribute to wide variation in outcomes both within and between centers. Identification of latent physiologic risk factors may strengthen predictive models. Therefore, more reliable tools to predict outcomes are needed. As described herein, the use of pre-operative total cell-free DNA in patient samples has been found to correlate with outcome in pediatric cardiac surgeries requiring cardiopulmonary bypass (Examples 1-2).

Without wishing to be bound by theory, it is thought that elevated levels of pre-operative total cf-DNA, which functions as a biomarker for cellular injury as well as a pro-inflammatory cytokine, are associated with poor outcome following surgery (e.g., pediatric cardiac surgery requiring cardiopulmonary bypass).

Accordingly, aspects of the disclosure relate to methods of quantifying pre-operative total cell-free DNA (such as in ng/ml) in a sample in order to determine a risk in a subject, such as any one of those provided herein. As provided herein, early detection or monitoring of the state or condition of a subject, such as one with any one of the conditions provided herein or one that will have surgery, such as heart surgery (e.g., such as heart surgery with cardiopulmonary bypass) can facilitate treatment and/or monitoring and improve clinical outcomes. In some embodiments, the early detection or monitoring of a subject pre-operatively can be used to guide surgical decisions (e.g., whether to proceed with the surgery), monitoring decisions (e.g., post-operatively) and/or treatment decisions (e.g., post-operatively).

Thus, in one embodiment of any one of the methods provided herein the subject is one that is pre-operative (e.g., one that is scheduled to have surgery), and the risk determined from a pre-operative sample from the subject can help guide actions post-surgery. The subject, in some embodiments or any one of the methods provided herein, is an infant or pediatric subject (e.g., less than 18 years of age, less than 16 years of age, less than 14 years of age, less than 12 years of age, less than 10 years of age, less than 8 years of age, less than 6 years of age, less than 5 years of age, less than 4 years of age, less than 3 years of age, less than 2 years of age, less than 1 year of age, or less than 6 months of age).

As used herein, “cell-free DNA” (or cf-DNA) is DNA that is present outside of a cell, e.g., in the blood, plasma, serum, etc. of a subject. Without wishing to be bound by any particular theory or mechanism, it is believed that cf-DNA is released from cells, e.g., via apoptosis of the cells. “Total cell-free DNA” (or total cf-DNA) is the total amount of cf-DNA present in a sample. As used herein, the methods provided herein can be used to determine an amount of pre-operative total cell-free DNA and a subject's risk of complications associated with, or following, a procedure (e.g., a heart surgery). Examples of complications include, but are not limited to, death, cardiac arrest, prolonged ventilation, prolonged length of stay in the hospital, infection, and requirement of mechanical circulatory support (e.g., extracorporeal membrane oxygenation, ECMO).

A subject may be assessed by determining or obtaining one or more amounts of cf-DNA as described herein. An amount of cf-DNA may be determined with experimental techniques, such as those provided elsewhere herein. An amount of pre-operative total cf-DNA can be “obtained” by any one of the methods provided herein or otherwise known in the art, and any obtaining step(s) can include any one of the methods incorporated herein by reference or otherwise provided herein. “Obtaining” as used herein refers to any method by which the respective information or materials can be acquired. Thus, the respective information can be acquired by experimental methods. An amount of cf-DNA (DS and/or total) may be determined with experimental techniques, such as those provided elsewhere herein or otherwise known in the art. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

In some embodiments, the sample may be taken after induction of anesthesia, prior to surgical incision. In other embodiments, the sample is taken 1, 2, 3, 4, 5, 6, 7, 8, 9, 10, 11, 12, or more hours prior to induction of anesthesia. In still further embodiments, for example, when monitoring a subject prior to surgery, a sample may be taken once, twice, thrice, or more frequently on a daily basis, or 1, 2, 3, 4, 5, 6, 7, or more days prior to surgery. In some embodiments, when monitoring the subject, samples may be taken 1, 2, 3, 4, 5 or more times a day for 1, 2, 3, 4, 5, 6, 7, or more days prior to surgery. In some embodiments, if the subject's levels of total cf-DNA are increasing, samples may be taken more frequently. In other embodiments, if the subject's levels of total cf-DNA are decreasing, samples may be taken less frequently.

Because of the ability to determine amounts of nucleic acids, such as cf-DNA, and the correlation with health, conditions and/or outcomes in a subject, the methods provided herein can be used to assess subjects. Thus, a risk of improving or worsening following a surgery can be determined in such subjects. A “risk” as provided herein, refers to the presence or absence or progression of any undesirable state or condition in a subject, or an increased likelihood of the presence or absence or progression of such a state or condition. As provided herein “increased risk” refers to the presence or progression of any undesirable state or condition in a subject or an increased likelihood of the presence or progression of such a state or condition. As an example, early detection of a risk can facilitate treatment and improve clinical outcomes (e.g., successful surgery). As provided herein, “decreased risk” refers to the absence of any undesirable state or condition or progression in a subject or a decreased likelihood of the presence or progression (or increased likelihood of the absence or nonprogression) of such a state or condition. The risk in any one of the methods provided herein may be the presence or progression of any one of the conditions or states provided herein.

Accordingly, in some embodiments of any one of the methods provided, the subject will undergo surgery (e.g., open heart surgery), and the risk is a risk associated with that surgery. In some embodiments of any one of the methods provided, the risk associated with the surgery is risk of death or complications, such as cardiac arrest, prolonged ventilation, prolonged length of stay in the hospital, infection, and requirement of mechanical circulatory support (e.g., extracorporeal membrane oxygenation, ECMO. In such an embodiment, the methods provided herein can be used to monitor a subject for worsening or improving condition. Thus, any one of the methods provided herein can include monitoring the subject more frequently or treating or modifying a treatment for a subject.

As provided herein, early detection or monitoring can facilitate treatment and improve clinical outcomes in the subjects as provided herein. Any one of the methods provided can be performed on any one of the subjects provided herein. Such methods can be used to monitor a subject over time (e.g., starting prior to surgery). In one embodiment of any one of the methods provided herein, the subject may be one that a clinician believes there is a likelihood of having a post-operative complication. In some embodiments, any one of the methods can be used to assess a subject that is at risk of having a post-operative complication. Subjects may be determined to have a likelihood or risk of having a post-operative complication based on symptoms (and/or lack thereof) prior to surgery. However, in some embodiments, the subject is determined to have a likelihood or risk of having a post-operative complication based on one or more other tests. In such an embodiment, the methods provided herein can be used to confirm such a finding or monitor such a subject for worsening or improving condition. Further, such methods can aid in the timing of a treatment or therapy or guide further treatment (e.g., alternative or intensified treatment, depending on risk level). Accordingly, the methods provided herein can be used to determine a treatment or monitoring regimen. Any one of the methods provided herein can comprise steps of determining a treatment and/or monitoring regimen.

“Determining a treatment regimen”, as used herein, refers to the determination of a course of action for treatment of the subject. In one embodiment of any one of the methods provided herein, determining a treatment regimen includes determining an appropriate therapy or information regarding an appropriate therapy to provide to a subject. In some embodiments of any one of the methods provided herein, the treatment regimen may comprise delaying a surgery or performing a surgery earlier. In some embodiments of any one of the methods provided herein, the determining includes providing an appropriate therapy or information regarding an appropriate therapy to a subject. As used herein, information regarding a treatment or therapy or monitoring may be provided in written form or electronic form. In some embodiments, the information may be provided as computer-readable instructions. In some embodiments, the information may be provided orally.

The therapies can be, for example, for treating any one of the conditions or states provided herein. Suitable therapies are provided or are known to those of ordinary skill in the art.

The therapies can also include, for example, immunosuppressives, plasmapheresis/plasma exchange, intravenous immunoglobulin, corticosteroids, anti-lymphocyte antibodies, and splenectomy. The therapies can also include, for example, retransplantation, percutaneous coronary interventions (PCI), coronary artery bypass grafting (CABG), transmyocardial laser revascularization and/or heparin-induced/mediated extracorporeal LDL plasmapheresis (HELP), as well as the administration of statins, anti-hypertensive agents, and/or anti-cytomegalovirus (anti-CMV) agents.

In another embodiment, the treatment can be a treatment for infection. In some embodiments, therapies for treating infection include therapies for treating a bacterial, fungal and/or viral infection. Such therapies include antibiotics. Other examples include, but are not limited to, amebicides, aminoglycosides, anthelmintics, antifungals, azole antifungals, echinocandins, polyenes, diarylquinolines, hydrazide derivatives, nicotinic acid derivatives, rifamycin derivatives, streptomyces derivatives, antiviral agents, chemokine receptor antagonist, integrase strand transfer inhibitor, neuraminidase inhibitors, NNRTIs, NS5A inhibitors, nucleoside reverse transcriptase inhibitors (NRTIs), protease inhibitors, purine nucleosides, carbapenems, cephalosporins, glycylcyclines, leprostatics, lincomycin derivatives, macrolide derivatives, ketolides, macrolides, oxazolidinone antibiotics, penicillins, beta-lactamase inhibitors, quinolones, sulfonamides, and tetracyclines

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Compositions for different routes of administration are known in the art (see, e.g., Remington's Pharmaceutical Sciences by E. W. Martin).

“Determining a monitoring regimen”, as used herein, refers to determining a course of action to monitor a state or condition in the subject over time. In one embodiment of any one of the methods provided herein, determining a monitoring regimen includes determining an appropriate course of action for determining the amount of total cf-DNA in the subject over time or at a subsequent point in time, or suggesting such monitoring to the subject. This can allow for the measurement of variations in a clinical state and/or permit calculation of normal values or baseline levels (as well as comparisons thereto). In some embodiments of any one of the methods provided herein determining a monitoring regimen includes determining the timing and/or frequency of obtaining samples from the subject and/or determining or obtaining an amount of total cf-DNA.

In some embodiments, amounts of total cf-DNA can be plotted over time. In some embodiments, threshold values for the points in time may also be plotted. For example, the threshold values can represent desirable or healthy values for the state or condition of a subject. Such plotting can be helpful to determine risk and/or to monitor a subject's progress. Such threshold values can be determined using data from a sufficient number of subjects. A comparison with a subject's cf-DNA level(s), such as pre-operative and/or post-operative, to such threshold values can be used to predict risk. Alternatively, whether or not total cf-DNA amounts increase or decrease over time in a subject can be used to predict risk and/or assess the state or condition of the subject.

Increasing or increased levels of total cf-DNA can correlate with increased risk; thus, a clinician may determine that a subject should undergo more frequent sampling if the subject's total cf-DNA is found to be increased and/or increasing between time points. If a subject is found to have decreased and/or decreasing levels of total cf-DNA between time points, a clinician may determine that less frequent sampling is sufficient. Additionally, if a subject does not show a decreased or decreasing level, such as below a threshold, the clinician may determine that additional testing and/or treatment and/or another type of treatment may be necessary. Steps of performing any one or more of the foregoing may be included in any one of the methods provided herein. Timing and/or frequency of monitoring may also be determined by a comparison to threshold values or other amount(s), such as those determined pre-operative and/or at other point(s) in time.

In some embodiments of any one of the methods provided herein, each amount and time point may be recorded in a report or in a database. Threshold values may also be recorded in a report or in a database.

Reports with any one or more of the values as provided herein are also provided in an aspect. Reports may be in oral, written (or hard copy) or electronic form, such as in a form that can be visualized or displayed. Preferably, the report provides the amount of total cf-DNA in a sample. In some embodiments, the report provides amounts of total cf-DNA in samples from a subject pre-operative and/or over time, and can further include corresponding threshold values in some embodiments.

In some embodiments, the amounts and/or threshold values are in or entered into a database. In one aspect, a database with such amounts and/or values is provided. From the amount(s), a clinician may assess the need for a treatment or monitoring of a subject. Accordingly, in any one of the methods provided herein, the method can include assessing the amount of total cf-DNA in the subject pre-operative and/or at more than one point in time. Such assessing can be performed with any one of the methods provided herein.

In some aspects, the methods include steps for determining a value for the amount of total cell-free nucleic acids (such as DNA), a value for the amount of specific cell-free nucleic acids (such as DNA) and/or a product of such values. As used herein, a “value” is any indicator that conveys information about an “amount”. The indicator can be an absolute or relative value for the amount. Further, the value can be the amount, frequency, ratio, percentage, etc. As used herein, the term “level” can be used instead of “amount” but is intended to refer to the same types of values.

In some embodiments, any one of the methods provided herein can comprise comparing an amount of total cf-DNA to a threshold value, such as pre-operative and/or to one or more subsequent amounts, to identify a subject at increased or decreased risk. In some embodiments of any one of the methods provided herein, a subject having an increased amount of total cf-DNA compared to a threshold value, such as pre-operative and/or to one or more subsequent amounts, is identified as being at increased risk. In some embodiments of any one of the methods provided herein, a subject having a decreased or similar amount of total cf-DNA compared to a threshold value, such as pre-operative and/or to one or more subsequent amounts, is identified as being at decreased or not increased risk.

“Threshold” or “threshold value”, as used herein, refers to any predetermined level or range of levels that is indicative of the presence or absence or progression of a state or condition or the presence or absence of a risk associated therewith. The threshold values can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a state or condition or risk or after a course of treatment. Such a baseline can be indicative of a normal or other state in the subject not correlated with the risk or state or condition that is being tested for. In some embodiments, the threshold value can be a baseline value of the subject being tested. Accordingly, the predetermined values selected may take into account the category in which the subject falls. Appropriate ranges and categories can be selected with no more than routine experimentation by those of ordinary skill in the art. The threshold values can be used for comparisons to make treatment and/or monitoring decisions. The determination can be done based on any one of the comparisons as provided herein with or without other indicators of risk or the state or condition of the subject. The threshold value of any one of the methods, reports, databases, etc. provided herein, can be any one of the threshold values provided herein, such as in the Examples or Figures.

The threshold values provided herein can be used to determine a risk level to a subject, in an embodiment of any one of the methods provided herein.

In some embodiments of any one of the methods provided herein, the pre-operative total cf-DNA level indicates increased or decreased risk. As provided herein “increased risk” refers to the presence or progression of any undesirable condition or state in a subject or an increased likelihood of the presence or progression of such a condition or state. As provided herein, “decreased risk” refers to the absence of any undesirable condition or state or progression in a subject or a decreased likelihood of the presence or progression (or increased likelihood of the absence or non-progression) of such a condition or state. Subjects at increased risk are likely to have complications following surgery, for example, may require a longer length of stay in the hospital and/or longer time on a ventilator. In an embodiment of any one of the methods provided herein the threshold value is 15, 16, 17 (e.g., 17.1), 18, 19 or 20 ng/ml. In an embodiment of any one of such methods, a subject with a value greater than a threshold may then be selected for treatment and/or further monitoring (e.g., delayed or canceled surgery) in any one of the methods provided herein. In an embodiment of any one of such methods provided herein, the method includes a step of further monitoring or treatment of the subject.

As described above, the level of pre-operative total cf-DNA may be used as a marker for risk. In some embodiments, the level of pre-operative total cf-DNA is used as an indicator of absolute risk; that is, near-term risk of poor clinical outcome, condition or state (e.g., following surgery).

The threshold values can also be used for comparisons to make treatment and/or monitoring decisions. For example, if the amount of pre-operative total cf-DNA is equal to or greater than any one of the thresholds provided herein and/or increasing over time in any one of the methods provided herein, further monitoring and/or treatment may be indicated.

Accordingly, any one of the methods provided herein may further include an additional test(s) for assessing the subject, or a step of suggesting such further testing to the subject (or providing information about such further testing). The additional test(s) may be any one of the methods provided herein. The additional test(s) may be any one of the other methods provided herein or otherwise known in the art as appropriate.

Exemplary additional tests for subjects, include, but are not limited to, echocardiogram, coronary angiography, intravascular ultrasound (IVUS), biopsy (e.g., endomycardial biopsy), stress echocardiography, CT coronary angiography, coronary flow reserve assessment (contrast-enhanced echocardiography), stress myocardial perfusion scintigraphy, positron emission tomography (PET) scanning, and measurement of serum biomarkers, such as BNP and/or troponin. In other embodiments of any one of the methods provided herein, the other test in addition to the level of BNP and/or troponin or in place thereof, is an echocardiogram.

Exemplary additional tests include, but are not limited to, positive C4d staining on biopsy (e.g., renal biopsy, endomycardial biopsy) and histopathological evidence of antibody-mediated injury (e.g., glomerulitis, peritubular capillaritis, arteritis).

Other examples of additional tests, include, but are not limited to, such as for subjects suspected of infection include, but are not limited to, blood tests, urine tests, throat swabs, and spinal tap.

The type of additional test(s) will depend upon the severity of the subject's condition and/or is well within the determination of the skilled artisan.

The amount of pre-operative total cf-DNA, may be determined by a number of methods. In some embodiments such a method is a sequencing-based method. In one embodiment, any one of the methods for determining pre-operative total cf-DNA may be any one of the methods of U.S. Publication No. 2015-0086477-A1, and such methods are incorporated herein by reference in their entirety.

An amount of cf-DNA may also be determined by a mismatch amplification-based assay, such as a MOMA assay. In one embodiment, any one of the methods for determining cf-DNA may be any one of the methods of PCT Publication No. WO 2016/176662 A1, and such methods are incorporated herein by reference in their entirety.

Other methods for determining total cell-free DNA in the subject are known in the art. In some embodiments of any one of the methods provided herein, the total cell-free DNA is determined with TAQMAN™ Real-time PCR using RNase P as a target or one or more other appropriate targets. Other methods would be apparent to those of ordinary skill in the art.

Any one of the methods provided herein can comprise extracting nucleic acids, such as cell-free DNA, from a sample obtained from a subject. Such extraction can be done using any method known in the art or as otherwise provided herein (see, e.g., Current Protocols in Molecular Biology, latest edition, or the QIAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 131′7-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

In some embodiments of any one of the methods provided herein, a pre-amplification step is performed. An exemplary method of such an amplification is as follows, and such a method can be included in any one of the methods provided herein. Approximately 15 ng of cell-free plasma DNA is amplified in a PCR using Q5 DNA polymerase with approximately 13 targets where pooled primers were at 4 uM total. Samples undergo approximately 25 cycles. Reactions are in 25 ul total. After amplification, samples can be cleaned up using several approaches including AMPURE bead cleanup, bead purification, or simply ExoSAP-IT™, or Zymo.

As used herein, the sample from a subject can be a biological sample. Examples of such biological samples include whole blood, plasma, serum, urine, etc. In some embodiments, addition of further nucleic acids, e.g., a standard, to the sample can be performed.

In another aspect, compositions and kits comprising one or more primer pairs as provided herein are provided. Other reagents for performing an assay, such as a PCR assay, may also be included in the composition or kit.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1

### Pre-Operative Total cf-DNA and Risk Stratification in Congenital Cardiac Surgery

A prospective observational study of children less than 18 years of age, and weighing more than 3 kg undergoing planned cardiopulmonary bypass (CPB) surgery was performed. A plasma total cell-free DNA (cf-DNA) sample was obtained after induction of anesthesia and prior to surgical incision. The primary outcome measure was a composite of post-operative cardiac arrest, ECMO, or death (CAED). Association of outcome to pre-operative total cf-DNA was assessed by logistic regression with a cutpoint chosen by ROC curve exploration. Odds ratios with 95% confidence intervals were also calculated.

Data were available in 117 patients, having a median age of 0.9 years (range 0-17.4), and a median weight of 7.8kg (range 3.2-98).

The primary outcome (CAED) was met in 6/117 (5.1%). Table 1 summarizes characteristics of patients with and without CAED. The risk of CAED was 2% with total cf-DNA<20 ng/ml, and 27% with total cf-DNA>20 ng/ml (OR=18.2, CI 2.2-212, p=0.002). Elevated total cf-DNA was associated with fewer hospital free days (GLM p<0.01). Data in Table 1 below is reported as median [IQR].

TABLE 1
Study Data

| | CAED | No CAED | Total | |
|---|---|---|---|---|
| | (n = 6) | (n = 111) | (n = 117) | P |
| Age (months) | 4.2 [0.9-9.3] | 13.2 [3.6-72] | 12 [3.6-67.2] | 0.07 |
| Weight (kg) | 4.6 [4.2-6.6] | 9.1 [5.1-17] | 7.8 [4.9-17] | 0.06 |
| CPB time | 4.0 [2.8-4.8] | 1.6 [1.2-2.4] | 1.6 [1.2- | <0.01 |
| (hours) | | | 2.6] | |
| TCF (ng/ml) | 25 [19-31] | 6.8 [3.9-11] | 6.9 [3.9-12] | 0.01 |
| TCF > 20 ng/ml | 4 (67%) | 11 (10%) | 15 (13%) | <0.01 |
| (N, %) | | | | |
| Hospital Free | 0 [0-0] | 7.6 [1.6-11] | 7.6 [0-11] | <0.01 |
| Days | | | | |

Pre-operative total cf-DNA was found to have an association with post-operative arrest, ECMO, and death.

Example 2

### Total Cell-free DNA Predicts Death and Adverse Events Following Pediatric Cardiothoracic Surgery

Congenital heart disease is the most common birth defect, affecting ˜1% of live births with approximately 30% requiring surgery. Total cell-free DNA (tcfDNA) is nuclear/genomic DNA released into the circulation due to cell turnover, apoptosis, tissue injury, fever, and illness.

A study was undertaken to measure tcfDNA in children undergoing congenital heart surgery with cardiopulmonary bypass (CPB). Elevated pre-operative tcfDNA was found to be associated with poor outcome following CPB.

### Methods

A prospective observational study of children <18 years and weight >3 kg undergoing planned cardiac surgery with CPB was performed. The primary outcome measure was a composite of post-operative cardiac arrest, the need for mechanical circulatory support, or death (CAED). Total cfDNA was measured as ng/mL plasma.

### Results

703 samples were collected from 117 patients undergoing 120 procedures. Median age: 0.9 years (0-17.4 years), median weight: 7.8 kg (3.2-98.0 kg). 6 patients met outcome (CAED; 6/117, 5.1%). All required mechanical circulatory support, one suffered cardiac arrest, and 3 died. Patients with CAED were younger: 0.35 years vs. 1.1 years (p=0.04). Patients with CAED were smaller: 4.7 kg vs. 8.9 kg (p=0.03).

### Conclusion

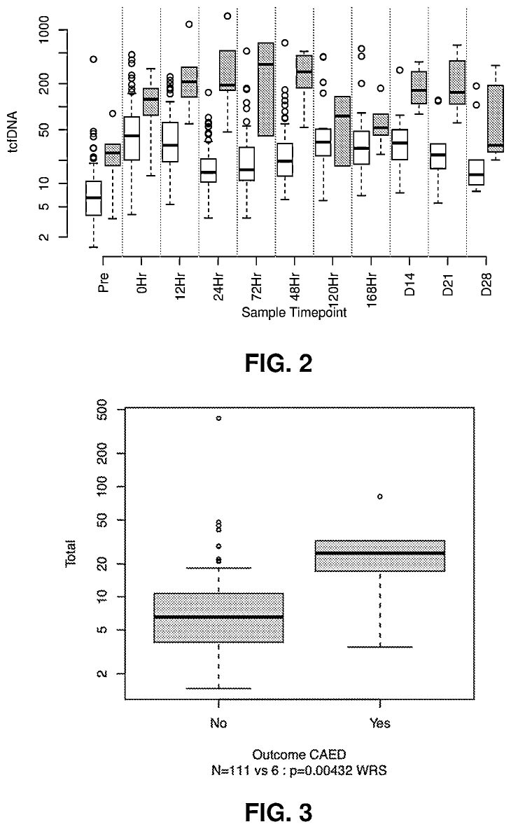

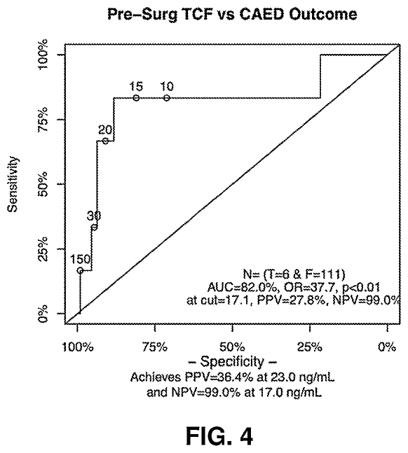

Elevated pre-operative tcfDNA levels were strongly associated with cardiac arrest, the need for mechanical circulatory support, or death following pediatric cardiac surgery with CPB (FIGS. 2-4). Total cf-DNA offers promise as a biomarker capable of predicting outcomes prior to clinical decompensation. Following the trend of tcfDNA post-operatively may prompt alternative or intensified treatment strategies in this vulnerable population. In this way, tcfDNA can be a tool to predict morbidity and mortality in children following surgery (e.g., cardiac surgery).

### Example 3

### Examples of Computer-Implemented Embodiments

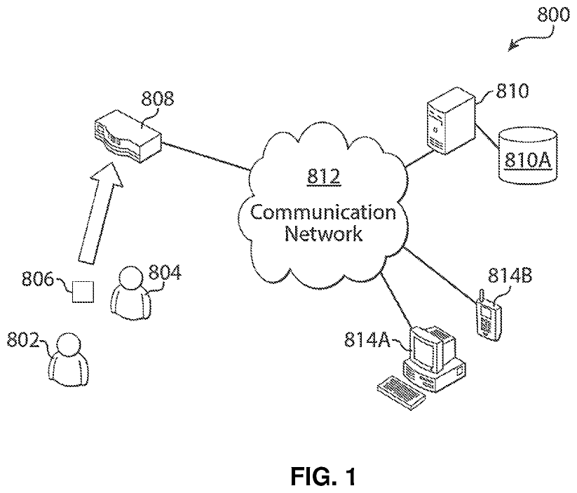

In some embodiments, the techniques described above may be implemented via one or more computing devices executing one or more software facilities to analyze samples for a subject over time, measure nucleic acids (such as cell-free DNA) in the samples, and produce a diagnostic result based on one or more of the samples. FIG. 1 illustrates an example of a computer system with which some embodiments may operate, though it should be appreciated that embodiments are not limited to operating with a system of the type illustrated in FIG. 1.

The computer system of FIG. 1 includes a subject 802 and a clinician 804 that may obtain a sample 806 from the subject 806. As should be appreciated from the foregoing, the sample 806 may be any suitable sample of biological material for the subject 802 that may be used to measure the presence of nucleic acids (such as cell-free DNA) in the subject 802, including a blood sample. The sample 806 may be provided to an analysis device 808, which one of ordinary skill will appreciate from the foregoing will analyze the sample 808 so as to determine (including estimate) a total amount of nucleic acids (such as cell-free DNA) in the sample 806 and/or the subject 802. For ease of illustration, the analysis device 808 is depicted as single device, but it should be appreciated that analysis device 808 may take any suitable form and may, in some embodiments, be implemented as multiple devices. To determine the amounts of nucleic acids (such as cell-free DNA) in the sample 806 and/or subject 802, the analysis device 808 may perform any of the techniques described above, and is not limited to performing any particular analysis. The analysis device 808 may include one or more processors to execute an analysis facility implemented in software, which may drive the processor(s) to operate other hardware and receive the results of tasks performed by the other hardware to determine on overall result of the analysis, which may be the amounts of nucleic acids (such as cell-free DNA) in the sample 806 and/or the subject 802. The analysis facility may be stored in one or more computer-readable storage media, such as a memory of the device 808. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

In some embodiments, the clinician 804 may directly provide the sample 806 to the analysis device 808 and may operate the device 808 in addition to obtaining the sample 806 from the subject 802, while in other embodiments the device 808 may be located geographically remote from the clinician 804 and subject 802 and the sample 806 may need to be shipped or otherwise transferred to a location of the analysis device 808. The sample 806 may in some embodiments be provided to the analysis device 808 together with (e.g., input via any suitable interface) an identifier for the sample 806 and/or the subject 802, for a date and/or time at which the sample 806 was obtained, or other information describing or identifying the sample 806.

The analysis device 808 may in some embodiments be configured to provide a result of the analysis performed on the sample 806 to a computing device 810, which may include a data store 810A that may be implemented as a database or other suitable data store. The computing device 810 may in some embodiments be implemented as one or more servers, including as one or more physical and/or virtual machines of a distributed computing platform such as a cloud service provider. In other embodiments, the device 810 may be implemented as a desktop or laptop personal computer, a smart mobile phone, a tablet computer, a special-purpose hardware device, or other computing device.

In some embodiments, the analysis device 808 may communicate the result of its analysis to the device 810 via one or more wired and/or wireless, local and/or wide-area computer communication networks, including the Internet. The result of the analysis may be communicated using any suitable protocol and may be communicated together with the information describing or identifying the sample 806, such as an identifier for the sample 806 and/or subject 802 or a date and/or time the sample 806 was obtained.

The computing device 810 may include one or more processors to execute a diagnostic facility implemented in software, which may drive the processor(s) to perform diagnostic techniques described herein. The diagnostic facility may be stored in one or more computer-readable storage media, such as a memory of the device 810. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

The diagnostic facility may receive the result of the analysis and the information describing or identifying the sample 806 and may store that information in the data store 810A. The information may be stored in the data store 810A in association with other information for the subject 802, such as in a case that information regarding prior samples for the subject 802 was previously received and stored by the diagnostic facility. The information regarding multiple samples may be associated using a common identifier, such as an identifier for the subject 802. In some cases, the data store 810A may include information for multiple different subjects.

The diagnostic facility may also be operated to analyze results of the analysis of one or more samples 806 for a particular subject 802, identified by user input, so as to determine a diagnosis for the subject 802. The diagnosis may be a conclusion of a risk that the subject 802 has, may have, or may in the future develop a particular condition or state or such a condition or state may worsen or progress. The diagnostic facility may determine the diagnosis using any of the various examples described above, including by comparing the amounts of nucleic acids (such as cell-free DNA) determined for a particular sample 806 to one or more thresholds or by comparing a change over time in the amounts of nucleic acids (such as cell-free DNA) determined for samples 806 over time to one or more thresholds. For example, the diagnostic facility may determine a risk to the subject 802 of a condition by comparing a total amount of nucleic acids (such as cell-free DNA) for one or more samples 806 to one threshold and comparing a total amount of nucleic acids (such as cell-free DNA) for one or more different sample(s) to another threshold or amount at another point in time(s). Based on the comparisons to the thresholds, the diagnostic facility may produce an output indicative of a risk to the subject 802.

As should be appreciated from the foregoing, in some embodiments, the diagnostic facility may be configured with different thresholds or other amounts to which amounts of nucleic acids (such as cell-free DNA) may be compared. The different thresholds may, for example, correspond to different demographic groups (age, gender, race, economic class, presence or absence of a particular procedure/condition/other in medical history, or other demographic categories), different conditions, and/or other parameters or combinations of parameters. In such embodiments, the diagnostic facility may be configured to select thresholds or other amounts against which amounts of nucleic acids (such as cell-free DNA) are to be compared, with different thresholds or other amounts stored in memory of the computing device 810. The selection may thus be based on demographic information for the subject 802 in embodiments in which thresholds differ based on demographic group, and in these cases demographic information for the subject 802 may be provided to the diagnostic facility or retrieved (from another computing device, or a data store that may be the same or different from the data store 810A, or from any other suitable source) by the diagnostic facility using an identifier for the subject 802. The selection may additionally or alternatively be based on the condition or state for which a risk is to be determined, and the diagnostic facility may prior to determining the risk receive as input a condition and use the condition or state to select the thresholds or other amounts on which to base the determination of risk. It should be appreciated that the diagnostic facility is not limited to selecting thresholds or other amounts in any particular manner, in embodiments in which multiple thresholds or other amounts are supported.

In some embodiments, the diagnostic facility may be configured to output for presentation to a user a user interface that includes a diagnosis of a risk and/or a basis for the diagnosis for a subject 802. The basis for the diagnosis may include, for example, amounts of nucleic acids (such as cell-free DNA) detected in one or more samples 806 for a subject 802. In some embodiments, user interfaces may include any of the examples of results, values, amounts, graphs, etc. discussed above. They can include results, values, amounts, etc. over time. In such a case, in some cases the graph may be annotated to indicate to a user how different regions of the graph may correspond to different diagnoses that may be produced from an analysis of data displayed in the graph. For example, thresholds or other amounts against which the graphed data may be compared to determine the analysis may be imposed on the graph(s).

A user interface including a graph, particularly with the lines and/or shading, may provide a user with a far more intuitive and faster-to-review interface to determine a risk of the subject 802 based on amounts of nucleic acids (such as cell-free DNA), than may be provided through other user interfaces. It should be appreciated, however, that embodiments are not limited to being implemented with any particular user interface.

In some embodiments, the diagnostic facility may output the diagnosis or a user interface to one or more other computing devices 814 (including devices 814A, 814B) that may be operated by the subject 802 and/or a clinician, which may be the clinician 804 or another clinician. The diagnostic facility may transmit the diagnosis and/or user interface to the device 814 via the network(s) 812.

Techniques operating according to the principles described herein may be implemented in any suitable manner. Included in the discussion above are a series of flow charts showing the steps and acts of various processes that determine a risk of a condition based on an analysis of amounts of nucleic acids (such as cell-free DNA). The processing and decision blocks discussed above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that embodiments are not limited to any particular syntax or operation of any particular circuit or of any particular programming language or type of programming language. Rather, one skilled in the art may use the description above to fabricate circuits or to implement computer software algorithms to perform the processing of a particular apparatus carrying out the types of techniques described herein. It should also be appreciated that, unless otherwise indicated herein, the particular sequence of steps and/or acts described above is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as a portion of a computing device or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Any one of the aforementioned, including the aforementioned devices, systems, embodiments, methods, techniques, algorithms, media, hardware, software, interfaces, processors, displays, networks, inputs, outputs or any combination thereof are provided herein in other aspects.

What is claimed is:

1. A method of assessing a risk in a subject, the method comprising:
(a) obtaining an amount of pre-operative total cell-free DNA (cf-DNA) in a sample from the subject; and
(b) optionally, reporting and/or recording the amount of pre-operative total cf-DNA.

2. The method of claim 1, wherein the method further comprises:
(c) comparing the amount of pre-operative total cf-DNA to a threshold total cf-DNA value or other amount from a different point in time.

3. The method of claim 1 or 2, wherein the method further comprises:
(d) determining a risk in the subject based on the obtained amount of pre-operative total cf-DNA or the comparison to the threshold or other total cf-DNA value.

4. The method of any one of the preceding claims, wherein the method further comprises:
(e) obtaining an amount of pre-operative total cf-DNA in one or more additional samples from the subject, each taken at different point in time.

5. The method of claim 4, wherein the different point(s) in time are one or more of any of the different points in time provided herein.

6. The method of claim 5, wherein the method further comprises:
(f) comparing the amount(s) of pre-operative total cf-DNA to threshold values or amounts from one or more different points in time of (e).

7. The method of claim 6, wherein the method further comprises:
(g) determining a risk in the subject based on a comparison(s) of (f).

8. The method of any one of the preceding claims, wherein the method further comprises:
(h) obtaining an amount of post-operative total cf-DNA in one or more additional samples from the subject, each taken at different point in time.

9. The method of claim 8, wherein the post-operative different point(s) in time are one or more of any of the different points in time provided herein, such as one or more or all of the following post-operative times: immediately following surgery, 12 hours, 24 hours, 48 hours, 72 hours, 120 hours, 168 hours, 10 days, 14 days, 21 days, or 28 days.

10. The method of claim 9, wherein the method further comprises:
(i) comparing the amount(s) of post-operative total cf-DNA to threshold values or amounts from one or more prior points in time.

11. The method of claim 1, wherein the method further comprises:
(j) determining a risk in the subject based on a comparison(s) of (i).

12. The method of any one of the preceding claims, wherein the method further comprises:
determining a treatment or monitoring regimen for the subject based on the amount(s) of pre-operative total cf-DNA and/or post-operative total cf-DNA and/or the comparison(s).

13. The method of any one of the preceding claims, wherein pre-operative total cf-DNA and/or post-operative total cf-DNA is obtained from samples taken from the subject once, twice, or thrice daily.

14. The method of any one of the preceding claims, wherein the subject is a pediatric subject.

15. The method of any one of the preceding claims, wherein the subject is assessed for at least 1, 2, 3, 4, 5, 6, or 7 days prior to surgery.

16. The method of any one of the preceding claims, wherein the method further comprises providing or obtaining one or more threshold values or amount(s) from one or more different points in time.

17. The method of claim 16, wherein a threshold value is provided for each time point an amount of pre-operative total cf-DNA and/or post-operative total cf-DNA is obtained.

18. The method of any one of the preceding claims, wherein the method further comprises obtaining the sample(s) from the subject.

19. The method of any one of the preceding claims, wherein the amount(s) and/or threshold value(s) are provided in a report.

20. A report that comprises the amount(s) and/or threshold value(s) of any one of the preceding claims.

21. The method of any one of claims 1-19, wherein the amount(s) and/or threshold value(s) are recorded in a database.

22. A database that comprises the amount(s) and/or threshold value(s) of any one of the preceding claims.

23. The method of any one of claims 12-22, wherein the determining a monitoring regimen comprises obtaining amount(s) of total cf-DNA in the subject over time or at a subsequent point in time, or suggesting such monitoring to the subject.

24. The method of any one of the preceding claims, wherein the time between samples is decreased if the amount(s) of pre-operative total cf-DNA and/or post-operative total cf-DNA is increased relative to threshold(s) or amount(s) from earlier time point(s).

25. The method of any one of the preceding claims, wherein the time between samples is increased if the amount of pre-operative total cf-DNA and/or post-operative total cf-DNA is decreased relative to threshold(s) or amount(s) from earlier time point(s).

26. The method of any one of claims 12-25, wherein the determining a monitoring regimen comprises using or suggesting the use of one or more additional test(s) to assess the subject.

27. The method of any one of claims 12-26, wherein the determining a monitoring regimen comprises assessing or suggesting the assessment of the subject at one or more points in time, determining or suggesting the frequency or timing of the assessment, changing the frequency or timing of the assessment or suggesting such change.

28. The method of any one of claims 12-27, wherein the determining a treatment regimen comprises selecting or suggesting a treatment for the subject or changing the treatment of the subject or suggesting such change or changing the timing or frequency of the treatment of the subject or suggesting such change.

29. The method of any one of claims 12-28, wherein the determining a treatment regimen comprises treating the subject.

30. The method of any one of claims 12-29, wherein the determining a treatment regimen comprises providing information about a treatment to the subject.

31. The method of any one of the preceding claims, wherein the sample is a blood, plasma or serum sample.

32. The method of any one of the preceding claims, wherein the subject is a non-transplant subject.

33. The method of any one of the preceding claims, wherein the subject is a cardiac surgery subject.

34. The method of any one of the preceding claims, wherein the subject is any one of the subjects provided herein.

35. The method of any one of the preceding claims, wherein the risk is a risk of cardiac arrest, need for mechanical ventilation, or death following surgery.

36. The method of any one of the preceding claims, wherein the risk is of any one of the states or conditions or outcomes as provided herein.

37. The method of any one of the preceding claims, wherein the threshold(s) is/are any one of the threshold value(s) as provided herein, such as 15, 16, 17 (e.g., 17.1), 18, 19, or 20 ng/mL.

38. The method of any one of the preceding claims, wherein the subject is monitored over time, such as with any one of the monitoring regimens as provided herein.

39. The method of any one of the preceding claims, wherein the subject is treated or treated over time, such as with any one of the treatments provided herein.

40. The method of any one of the preceding claims, wherein the treatment or monitoring regimen is suggested to, prescribed for, given to or performed on the subject.
